(19) United States
(12) Patent Application Publication
(10) Pub. No.: US 2013/0310711 A1
(43) Pub. Date: Nov. 21, 2013

(54) JOINT MOTION MEASURING APPARATUS AND MEASURING METHOD THEREOF

(71) Applicant: NATIONAL CHENG KUNG UNIVERSITY, Tainan City (TW)

(72) Inventors: Jeen-Shing WANG, Tainan City (TW); Ming-Hsin YEN, Tainan City (TW); Yu-Liang HSU, Kaohsiung City (TW)

(73) Assignee: NATIONAL CHENG KUNG UNIVERSITY, Tainan City (TW)

(21) Appl. No.: 13/830,836

(22) Filed: Mar. 14, 2013

(30) Foreign Application Priority Data
May 18, 2012 (TW) 101117864

(51) Int. Cl. A61B 5/11; A61B 5/00

(52) U.S. Cl. CPC A61B 5/1121; A61B 5/4528; A61B 5/72; A61B 5/1126; A61B 5/6801
USPC 600/595

(57) ABSTRACT

A joint motion measuring apparatus is applied for measuring a rotation angle of a joint. A human body has a moving part connected with the joint. The joint motion measuring apparatus includes an attitude sensing unit, an attitude computing unit, and a joint motion computing unit. The attitude sensing unit is placed on the moving part, and senses the moving part moving from a first position to a second position to output a motion sensing signal. The attitude computing unit is coupled with the attitude sensing unit and transfers the motion sensing signal into an attitude signal. The joint motion computing unit is coupled with the attitude computing unit, and computes a zenith angle of the attitude sensing unit at the second position according to the attitude signal to further obtain the rotation angle of the joint according to the zenith angle.

### CROSS REFERENCE TO RELATED APPLICATIONS

This Non-provisional application claims priority under 35 U.S.C. §119(a) on Patent Application No(s). 101117864 filed in Taiwan, Republic of China on May 18, 2012, the entire contents of which are hereby incorporated by reference.

### BACKGROUND OF THE INVENTION

1. Field of Invention

The invention relates to a measuring apparatus and a measuring method thereof and, in particular, to a joint motion measuring apparatus and a measuring method thereof.

2. Related Art

Because of the aging of population and the change of life styles, many chronic diseases (e.g. stroke, frozen shoulder, osteoarthritis, or degenerative arthritis) have become an important issue. These chronic diseases not only cause patients much pain, but also affect the range of motion (ROM) of the patient's joint. Besides, these diseases hinder the patient from normal activity.

For the clinical diagnosis, the doctor needs to assess the degree of impairment of joint movement according to the range of motion (ROM) evaluation, and then provide appropriate therapy for the patient. The patients suffering from these chronic diseases can recover their functional ability and improve their quality of life through physiotherapy rehabilitation. During the rehabilitation process, clinicians or physical therapists can evaluate patient's condition changes through measuring the range of motion of the patient's joint. Therefore, it can be seen that the measuring of the joint motion is an important indication of assessing the degree of impairment of the joint for clinicians or physical therapists.

Nowadays, the range of motion of joint is measured manually by a universal goniometer. However, this not only consumes a lot of time, but needs auxiliary manpower to facilitate the measuring process for obtaining an accurate ROM. Moreover, the universal goniometer suffers from low accuracy due to repeated measurement variation which are affected by the experiences of testers (clinicians or physical therapists), or different measuring period. In other words, different doctors or therapists will obtain different ROM results for the same patient with the same rotation angle of joint by using the universal goniometer. Even the same patient with the same rotation angle of the joint measured by the same clinician or therapist can acquire different range of motion of joint at different times. Therefore, the universal goniometer easily causes considerable measurement error that even achieves 10° or more. So, the electrogoniometer or electronic inclinometer has been developed recently for avoiding the drawbacks of the universal goniometer. Although the electrogoniometer or electronic inclinometer has an advantage of decreasing the required time of measuring the joint motion, it still needs to be handled by a professional therapist for performing the ROM measuring. Besides, measurement error is generated by the different locations where the electrogoniometer or electronic inclinometer is placed, the different measuring experiences of the clinicians or therapists, or the different measuring period.

FIGS. 1A to 1C are schematic diagrams in which the range of motion of the shoulder joint is measured by an electrogoniometer or an electronic inclinometer 1.

As shown in FIGS. 1A and 1B, in the normal measurement, the measured arm moves frontward from the position as shown in FIG. 1A to the position as shown in FIG. 1B. But, if the measured arm rotates during the frontward movement as shown in FIG. 1C, the muscle of the upper arm will generate involuntary rotation to cause the electrogoniometer or electronic inclinometer 1 to be deviated, resulting in the inaccurate measuring. Besides, if the measured arm is lifted with leftward or rightward deviation rather than vertical movement during the frontward movement, the accuracy provided by the electrogoniometer or electronic inclinometer 1 will be decreased.

Therefore, it is an important issue to provide a joint motion (range of motion) measuring apparatus and a measuring method thereof that can overcome the measurement error caused by the muscle rotation or the deviation of the movement during the measurement of the range of motion of the joint for obtaining better accuracy.

### SUMMARY OF THE INVENTION

In view of the foregoing subject, an objective of the invention is to provide a joint motion measuring apparatus and a measuring method thereof that can overcome the measurement error caused by the muscle rotation or the deviation of the movement during the measurement of the range of motion of the joint for obtaining better accuracy.

To achieve the above objective, a joint motion measuring apparatus according to the invention is applied for measuring a rotation angle of a joint. A human body has a moving part connected with the joint. The joint motion measuring apparatus includes an attitude sensing unit, an attitude computing unit, and a joint motion computing unit. The attitude sensing unit is placed on the moving part, and senses the moving part moving from a first position to a second position to output a motion sensing signal. The attitude computing unit is coupled with the attitude sensing unit, and transfers the motion sensing signal into an attitude signal. The joint motion computing unit is coupled with the attitude computing unit, and computes a zenith angle of the attitude sensing unit at the second position according to the attitude signal to further obtain the rotation angle of the joint.

In one embodiment, the joint motion measuring apparatus is wearable to be placed on the moving part.

In one embodiment, the attitude sensing unit includes a gyroscope, an accelerometer, a magnetometer, or an electronic compass, or their any combination.

In one embodiment, the motion sensing signal includes an angular velocity, an acceleration, a magnetic field intensity, a geomagnetic azimuth, or their any combination, caused by the location changes of the moving part moving from the first position to the second position.

In one embodiment, the attitude computing unit integrates the angular velocity sensed by a triaxial gyroscope to obtain an orientation angle.

In one embodiment, the attitude computing unit obtains an orientation angle according to the triaxial gravity components sensed by an accelerometer.

In one embodiment, the attitude computing unit obtains an orientation angle according to the magnetic strength sensed by a triaxial magnetometer.

In one embodiment, the attitude computing unit obtains an orientation angle according to a geomagnetic azimuth sensed by an electronic compass,

In one embodiment, the attitude computing unit obtains an orientation angle according to the angular velocity, the acceleration, the magnetic field intensity, the geomagnetic azimuth, or their any combination.

In one embodiment, the joint motion computing unit receives the orientation angle to generate a transformation matrix.

In one embodiment, the joint motion computing unit multiplies the transformation matrix by a first position vector of the attitude sensing unit at the first position to obtain a second position vector of the attitude sensing unit at the second position, and thus obtain the zenith angle.

In one embodiment, the rotation angle of the joint is equal to an ideal angle of the range of motion of the joint minus the zenith angle.

In one embodiment, the rotation angle of the joint is equal to the zenith angle.

To achieve the above objective, a joint motion measuring method according to the invention is cooperated with a joint motion measuring apparatus and applied for measuring a rotation angle of a joint of a human body having a moving part connected with the joint. The joint motion measuring apparatus includes an attitude sensing unit, an attitude computing unit and a joint motion computing unit. The joint motion measuring method comprises steps of: sensing the moving part moving from a first position to a second position to output a motion sensing signal by the attitude sensing unit; transferring the motion sensing signal into an attitude signal by the attitude computing unit; and computing a zenith angle of the attitude sensing unit at the second position according to the attitude signal to further obtain the rotation angle of the joint by the joint motion computing unit.

In one embodiment, the joint motion measuring method further comprises receiving the orientation angle to generate a transformation matrix by the joint motion computing unit.

In one embodiment, the joint motion measuring method further comprises multiplying the transformation matrix by a first position vector of the attitude sensing unit at the first position to obtain a second position vector of the attitude sensing unit at the second position to further obtain the zenith angle by the joint motion computing unit.

As mentioned above, in the joint motion measuring apparatus and the measuring method thereof according to the invention, the attitude sensing unit senses the moving part (connected with the joint to be measured) moving from a first position to a second position to output a motion sensing signal, and the attitude computing unit transfers the motion sensing signal into an attitude signal, and then the joint motion computing unit can compute a zenith angle of the attitude sensing unit at the second position according to the attitude signal to further obtain the rotation angle of the joint. Thereby, the measurement error caused by the muscle rotation or the deviation of the movement during the measurement of the range of motion of the joint can be overcome for obtaining better accuracy.

### BRIEF DESCRIPTION OF THE DRAWINGS

The invention will become more fully understood from the detailed description and accompanying drawings, which are given for illustration only, and thus are not limitative of the present invention, and wherein:

FIGS. 1A to 1C are schematic diagrams in which the range of motion of the shoulder joint is measured by a conventional electrogoniometer or a conventional electronic inclinometer;

FIG. 2A is a schematic diagram showing the measurement performed by a joint motion measuring apparatus according to a preferred embodiment of the invention;

FIG. 2B is a block diagram of the joint motion measuring apparatus according to a preferred embodiment of the invention;

FIG. 3 is a schematic diagram showing triaxial angular velocities of the joint motion measuring apparatus according to a preferred embodiment of the invention;

FIG. 4 is a schematic diagram showing a position of a spherical coordinate system relatively in a rectangular coordinate system;

FIG. 5A is a schematic top-view of the flexion motion of the shoulder joint measured by the joint motion measuring apparatus in FIG. 2A;

FIGS. 5B and 5C are other side-views of the shoulder joint measured by the joint motion measuring apparatus;

FIG. 6 is a block diagram of another joint motion measuring apparatus according to a preferred embodiment of the invention; and

FIG. 7 is a flow chart of a joint motion measuring method according to a preferred embodiment of the invention.

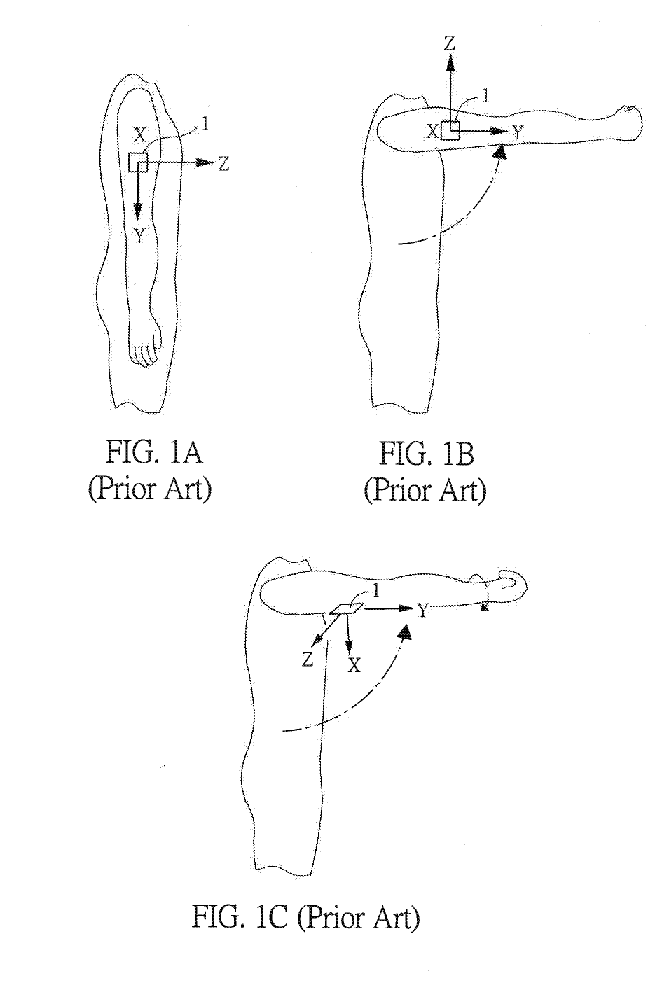

### DETAILED DESCRIPTION OF THE INVENTION

The present invention will be apparent from the following detailed description, which proceeds with reference to the accompanying drawings, wherein the same references relate to the same elements.

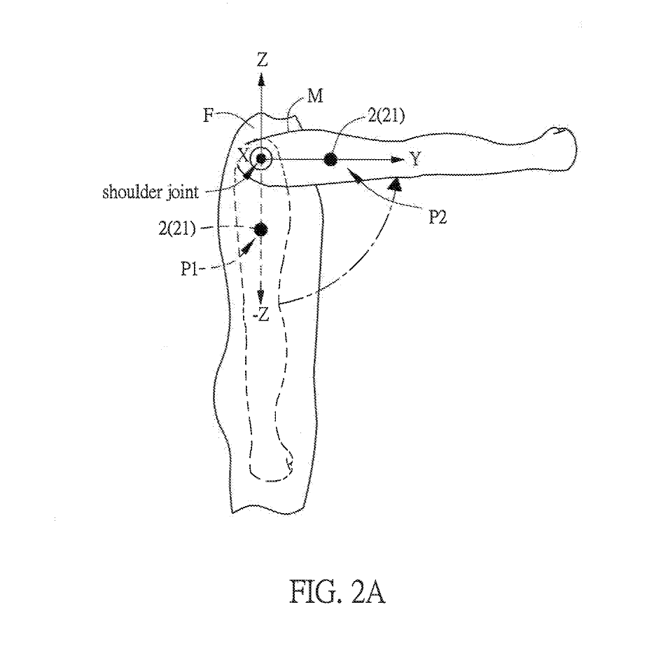

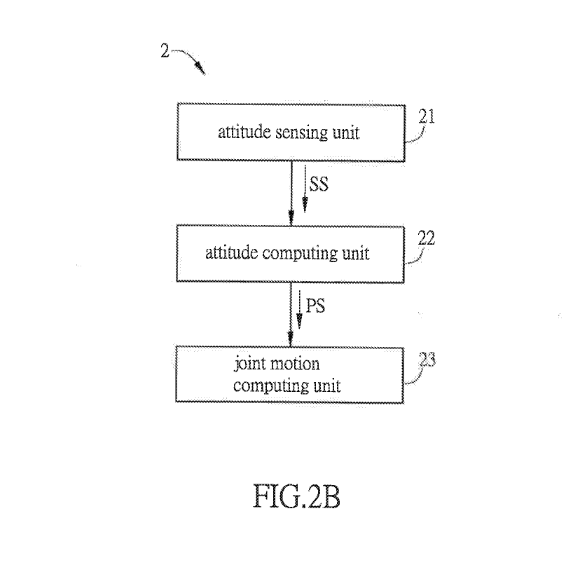

FIG. 2A is a schematic diagram showing the measurement performed by a joint motion measuring apparatus 2 of a preferred embodiment of the invention, and FIG. 2B is a block diagram of the joint motion measuring apparatus 2.

The joint motion measuring apparatus 2 of the invention can measure a rotation angle of a joint of a human body. The human body can have a fixed part F and a moving part M, and the fixed part F is connected with the moving part M through the joint. The joint motion measuring apparatus 2 can be placed on the moving part M. Specifically, taking an example of measuring the range of motion of the shoulder joint as shown in FIG. 2A, when the upper arm and the forearm moves straight, frontward, and upward, and the intersection (e.g. the acromion process) of the upper arm and the shoulder is fixed as a fixed point, such motion is called shoulder flexion exercise. In this case, the moving part M is defined as the middle portion of the upper arm connecting to the (shoulder) joint, and the fixed part F includes the shoulder and the trunk. Herein, the joint motion measuring apparatus 2 is placed to the middle portion of the upper arm (the moving part M). Otherwise, to take the knee joint as an example, the moving part M can be a lower leg connecting to the knee joint while the fixed part F is the thigh. However, the invention is not limited thereto. In the invention, the rotation angle of any joint can be measured, and then the related moving part M and fixed part F are ascertained accordingly. The joint motion measuring apparatus 2 can be made as a wearable type thus to be put on the moving part M (the upper arm). For example, the joint motion measuring apparatus 2 can be fixed on the moving part M (the upper arm) by an adhesive element (e.g. a velcro). Thereby, the measurement data will not be affected by the different measuring experiences of the clinicians or physical therapists, the different measuring period or the different disposition location.

The joint motion measuring apparatus 2 includes an attitude sensing unit 21, an attitude computing unit 22, and a joint motion computing unit 23.

As shown in FIGS. 2A and 2B, the attitude sensing unit 21 is placed on the moving part M, and can sense the moving part M moving from a first position P1 to a second position P2. Accordingly, the attitude sensing unit 21 can sense the upper arm moving from the first position P1 to the second position P2, and thus output a motion sensing signal SS. In this embodiment, as shown in FIG. 2A, the first position P1 is the position of the arm drooping towards the inverse direction of the Z axis, and the second position P2 is the position of the arm moving frontward and upward to the direction of the Y axis. As shown in FIG. 2A, the joint motion measuring apparatus 2 and the attitude sensing unit 21 are both placed on the moving part M. Otherwise, the attitude sensing unit 21 is placed on the moving part M while the attitude computing unit 22 and the joint motion computing unit 23, and/or other components are placed on other locations, and in this case, the motion sensing signal SS outputted by the attitude sensing unit 21 can be transmitted to the attitude computing unit 22 and the joint motion computing unit 23 by a wired or wireless method for the subsequent process.

The attitude sensing unit 21 can include, for example, a gyroscope, an accelerometer, a magnetometer, or an electronic compass, or their any combination. The aforementioned devices each can be a uniaxial or multiaxial device. Herein, a triaxial gyroscope is used, and it can sense triaxial angular velocities. The motion sensing signal SS can contain an angular velocity, an acceleration, a magnetic field intensity, a geomagnetic azimuth, or their any combination, caused by the location changes of the moving part M moving from the first position P to the second position P2, and they can be multiaxial signals. In other embodiments, the joint motion measuring apparatus further can include a filter unit (not shown), which can filter the noise of the inertia device (such as the aforementioned gyroscope, accelerometer, magnetometer, or electronic compass, or their any combination) of the attitude sensing unit 21, or filter the unwanted influence from the action (such as hand's shake) or from the surrounding environment which causes the motion sensing signal SS erroneous.

The attitude computing unit 22 is coupled with the attitude sensing unit 21, and able to transfer the motion sensing signal SS into an attitude signal PS. The said coupling can be achieved by a wired method, a wireless method, or their combination. Herein, the attitude computing unit 22 can integrate the triaxial angular velocities sensed by the triaxial gyroscope during the transition from the first position P1 to the second position P2, to obtain an orientation angle (e.g. attitude signal PS) of the rotating joint. Otherwise, the attitude computing unit 22 can obtain an orientation angle (i.e. represented by the attitude signal PS) of the rotating joint according to the triaxial gravity components caused by the location changes of an accelerometer from the first position P1 to the second position P2. Otherwise, the attitude computing unit 22 can obtain an orientation angle (represented by the attitude signal PS) of the rotating joint according to the triaxial magnetic strength sensed by the triaxial magnetometer moving from the first position P1 to the second position P2 or according to the geomagnetic azimuth sensed by the electronic compass. Accordingly, the attitude computing unit 22 can obtain an orientation angle of the joint motion measuring apparatus 2 according to the angular velocity, the acceleration, the magnetic field intensity, the geomagnetic azimuth, or their any combination, caused by the location changes of the joint motion measuring apparatus 2 from the first position P1 to the second position P2. Herein, for example, the attitude computing unit 22 obtains an orientation angle of the joint motion measuring apparatus 2 according to the angular velocity sensed by the attitude sensing unit 21 during the transition of the joint motion measuring apparatus 2 from the first position P1 to the second position P2.

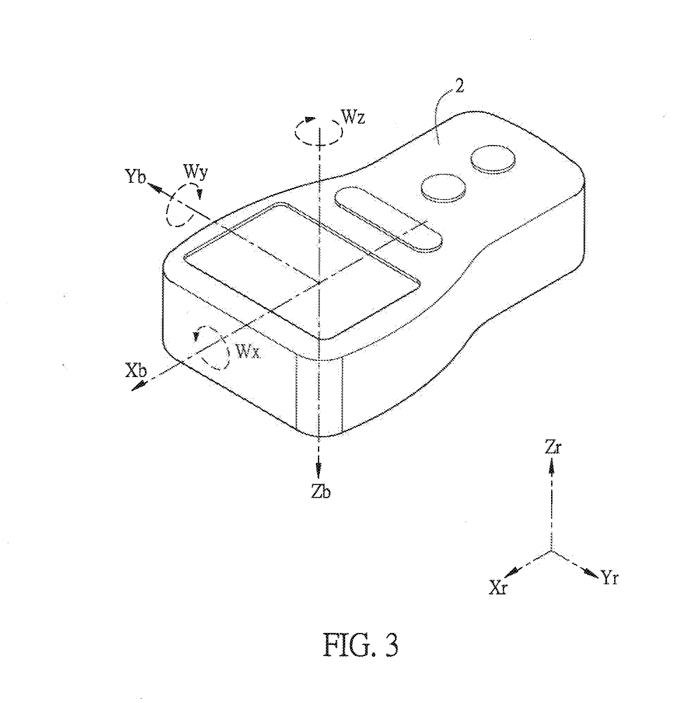

As shown in FIG. 3, the coordinates Xb, Yb, Zb represent a body coordinate system of the joint motion measuring apparatus 2, and the coordinates Xr, Yr, Zr represent a reference coordinate system. In addition, the aforementioned orientation angle can represent the relative angle or rotation of the joint motion measuring apparatus 2 between the body coordinate system and the reference coordinate system.

The orientation angle contains the roll angle (Φ), the pitch angle (θ), and the yaw angle (ψ). As shown in FIG. 3 (not showing roll angle (Φ), pitch angle (θ), and yaw angle (ψ)), the roll angle (Φ) represents the rotation angle of the joint motion measuring apparatus 2 on the axis Xb, and can be obtained by integrating the variation of the angular velocity Wx on the axis Xb sensed by the gyroscope (the attitude sensing unit 21) during the transition of the moving part M from the first position P1 to the second position P2. In other embodiments, the roll angle (Φ) can be obtained by using the variation of the triaxial gravity components sensed by the accelerometer (the attitude sensing unit 21) during the transition of the moving part M from the first position p1 to the second position P2. The pitch angle (θ) represents the rotation angle of the joint motion measuring apparatus 2 on the axis Yb, and can be obtained by integrating the variation of the angular velocity Wy on the axis Yb sensed by the gyroscope (the attitude sensing unit 21) during the transition of the moving part M from the first position P1 to the second position P2. In other embodiments, the pitch angle (θ) can be obtained by using the variation of the triaxial gravity components sensed by the accelerometer (the attitude sensing unit 21) during the transition of the moving part M from the first position p1 to the second position P2. The yaw angle (ψ) represents the rotation angle of the joint motion measuring apparatus 2 on the axis Zb, and can be obtained by integrating the variation of the angular velocity Wz on the axis Zb sensed by the gyroscope (the attitude sensing unit 21) during the transition of the moving part M from the first position P1 to the second position P2. In other embodiments, the yaw angle (ψ) can be obtained by using the variation of the magnetic field intensity sensed by the magnetometer (the attitude sensing unit 21) or the variation of the geomagnetic azimuth sensed by the electronic compass (the attitude sensing unit 21) during the transition of the moving part M from the first position P1 to the second position P2. Besides, the roll angle (Φ), the pitch angle (θ), and the yaw angle (ψ) can be obtained by the angular velocity, the acceleration, the magnetic field intensity, the geomagnetic azimuth, or their any combination sensed by the attitude sensing unit 21 during the transition of the moving part M from the first position P1 to the second position P2. However, the invention is not limited thereto.

In FIG. 2B, the joint motion computing unit 23 is coupled with the attitude computing unit 22, and can, according to the attitude signal PS (e.g. the orientation angle: the roll angle (Φ), the pitch angle (θ), and the yaw angle (ψ)), compute a zenith angle θ′ of the attitude sensing unit 21 at the second position P2 to further obtain the rotation angle of the joint.

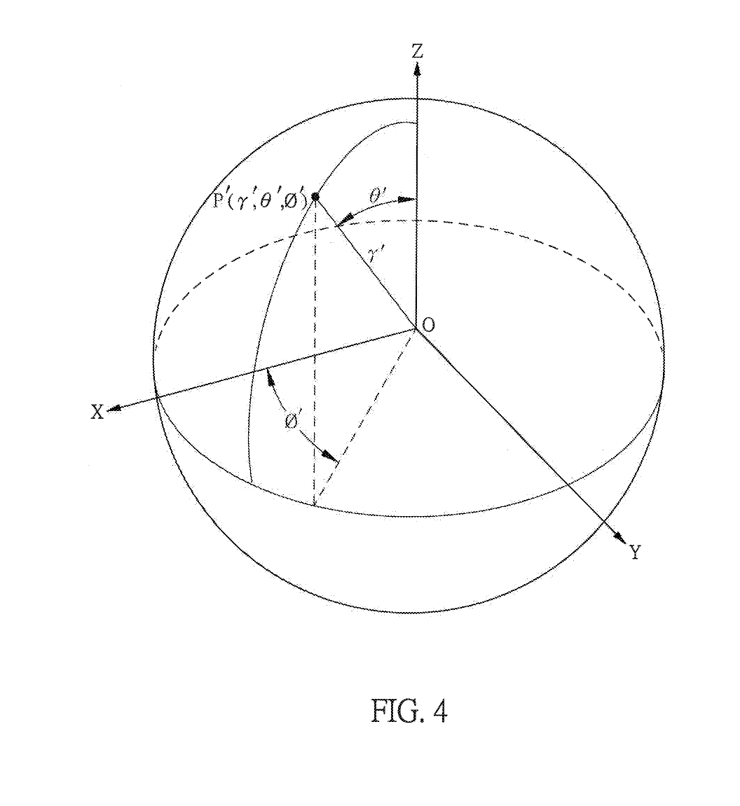

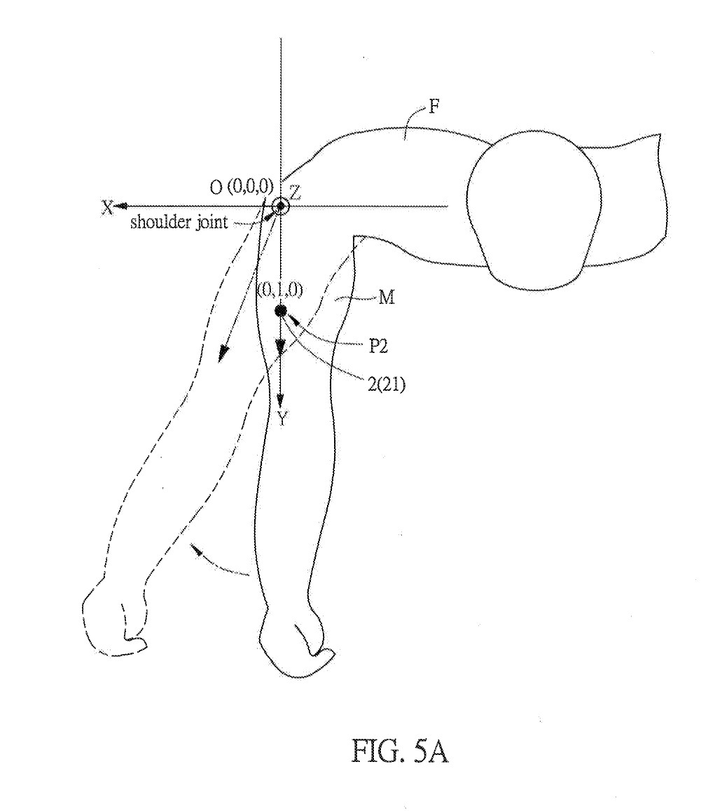

FIG. 4 is a schematic diagram showing a position P′(γ′, θ′, φ′) of a spherical coordinate system relatively in a rectangular coordinate system (X, Y, Z).

The position P′ of the spherical coordinate system is represented by a radial distance γ′, a zenith angle θ′, and an azimuth angle φ′. In FIG. 4, the distance between the origin O and the position P′ is the radial distance γ′, the included angle between the line through the origin O and position P′ and the Z axis is the zenith angle θ′, and the included angle between the projection line on the X-Y plane of the line through the origin O and position P′ and the X axis is the azimuth angle φ′. The conversion relationship between the set of the radial distance γ′, zenith angle θ′ and azimuth angle φ′ and the rectangular coordinates X, Y, Z is as the following equation (1):

$${γ}^{′} = \sqrt{{x}^{2} + {y}^{2} + {z}^{2}} , {θ}^{′} = arctan ( \frac{\sqrt{{x}^{2} + {y}^{2}}}{z} ) {φ}^{′} = arctan ( \frac{y}{x} ) \qquad ( 1 )$$

Besides, the joint motion computing unit 23 can receive the orientation angle outputted by the attitude computing unit 22 thus to generate a transformation matrix T as follows:

$$T ( t ) = [ \begin{matrix}cos ψ ( t ) & - sin ψ ( t ) & 0 \\ sin ψ ( t ) & cos ψ ( t ) & 0 \\ 0 & 0 & 1\end{matrix} ] [ \begin{matrix}cos θ ( t ) & 0 & sin θ ( t ) \\ 0 & 1 & 0 \\ - sin θ ( t ) & 0 & cos θ ( t )\end{matrix} ] [ \begin{matrix}1 & 0 & 0 \\ 0 & cos φ ( t ) & - sin φ ( t ) \\ 0 & sin φ ( t ) & cos φ ( t )\end{matrix} ] \qquad ( 2 )$$

Accordingly, the variation of the orientation angle in the three dimensional space at a certain time during the transition of the upper arm from the first position P1 to the second position P2 can be obtained by the attitude sensing unit 21 and the attitude computing unit 22, and then the transformation matrix T at the certain time can be obtained by the variation of the orientation angle. The joint motion computing unit 23 can further multiply the transformation matrix T by a first position vector V1 of the attitude sensing unit 21 at the first position P1 to obtain a second position vector V2 of the attitude sensing unit 21 at the second position P2, and thus obtain the zenith angle θ′. The aforementioned computation can be represented by the equation as follows:

V2=T×V1 (3)

In the equation (3), V1 is a position vector at a prior time (e.g. the time at the first position P1) in the rectangular coordinate system, and V2 is a position vector at a present time (e.g. the time at the second position P2) in the rectangular coordinate system.

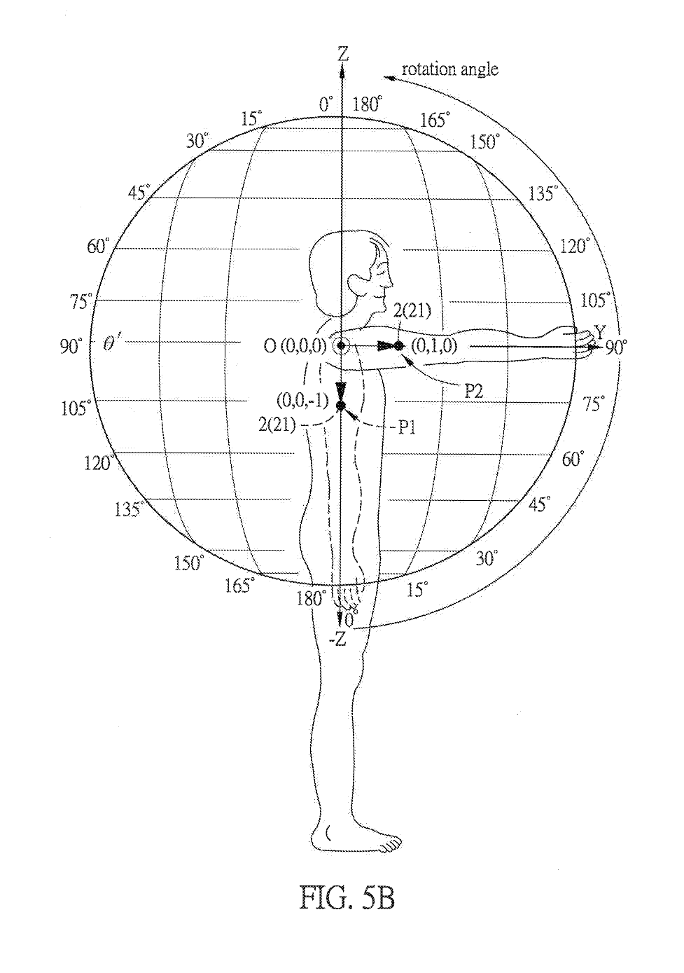

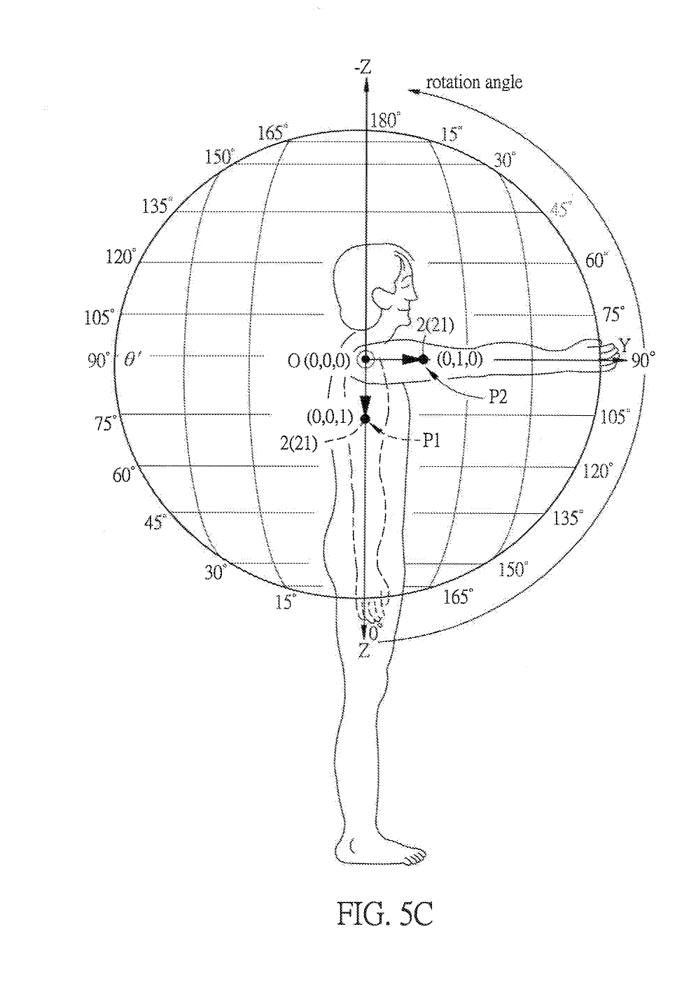

Finally, the zenith angle θ′ that has been obtained is used to obtain the rotation angle of the joint. The rotation angle of the joint is equal to the ideal angle of the range of motion of the joint minus the zenith angle θ′, or equal to the zenith angle θ′. Thereinafter, the measuring process and the computing process of the joint motion measuring apparatus 2 are illustrated by referring to FIG. 2A with FIGS. 5A and 5B for example.

FIG. 5A is a schematic top-view of the flexion motion of the shoulder joint measured by the joint motion measuring apparatus 2, and FIG. 5B is another side-view of the flexion motion of the shoulder joint measured by the joint motion measuring apparatus 2. Herein, the measurement is also performed to the flexion motion of the shoulder joint, and as shown in FIG. 5B, the upper arm still moves from the first position P1 to the second position P2.

In this embodiment, the vertical direction of the head of the patient is defined as the positive direction of the Z axis. At an initial time (i.e. the arm drooping at the first position P1), the flexion angle of the arm is zero while the initial roll angle (Φ), pitch angle (θ) and yaw angle (ψ) sensed by the attitude sensing unit 21 are all zero. On the assumption that the intersection (e.g. the acromion process) of the upper arm and the shoulder is a fixed origin O with the coordinates (0, 0, 0) and the middle location of the upper arm (i.e. where the attitude sensing unit 21 is placed for sensing the rang of motion of the joint) has the coordinates (0, 0, −1), the first position vector V1 can be derived as

$$[ \begin{matrix}0 \\ 0 \\ - 1\end{matrix} ]$$

from the coordinates of the two locations.

When the arm reaches the second position P2 from the first position P1, the variations of the roll angle (Φ), pitch angle (θ) and yaw angle (ψ) sensed by the attitude sensing unit 21 are 90°, 0°, 0°, respectively. By substituting the variations of the roll angle (Φ), pitch angle (θ) and yaw angle (ψ) into the equation (2), the transformation matrix T can be obtained as follows:

$$T = [ \begin{matrix}1 & 0 & 0 \\ 0 & 1 & 0 \\ 0 & 0 & 1\end{matrix} ] [ \begin{matrix}1 & 0 & 0 \\ 0 & 1 & 0 \\ 0 & 0 & 1\end{matrix} ] [ \begin{matrix}1 & 0 & 0 \\ 0 & 0 & - 1 \\ 0 & 1 & 0\end{matrix} ] = [ \begin{matrix}1 & 0 & 0 \\ 0 & 0 & - 1 \\ 0 & 1 & 0\end{matrix} ] \qquad ( 4 )$$

Then, the transformation matrix T is substituted into the equation (3), and thus the second position vector V2 of the second position P2 is equal to the product of the transformation matrix T and the first position vector V1 as follows:

$${V}_{2} = [ \begin{matrix}1 & 0 & 0 \\ 0 & 0 & - 1 \\ 0 & 1 & 0\end{matrix} ] [ \begin{matrix}0 \\ 0 \\ - 1\end{matrix} ] = [ \begin{matrix}0 \\ 1 \\ 0\end{matrix} ] \qquad ( 5 )$$

Accordingly, because the origin O with the coordinates (0, 0, 0) is fixed, the absolute location of the attitude sensing unit 21 in the rectangular coordinate system can be represented as (0, 1, 0). When the coordinates (0, 1, 0) (i.e. x=0, y=1, z=0) are substituted into the equation (1), the zenith angle θ′ can be obtained as 90°. Since the positive direction of the Z axis is set as the vertical direction of the patient's head in this embodiment, the rotation angle of the joint is equal to the ideal angle of the range of motion of the joint minus the zenith angle θ′. The aforementioned ideal angle of the range of motion of the joint means an ideal rotation angle of the joint of a normal human. In general, the ideal rotation angle of the shoulder joint during the flexion motion is 180°, so the rotation angle of the shoulder joint moving form the first position P1 to the second position P2 is equal to 90° (180°-90°). Accordingly, in the invention, the zenith angle θ′ of the moving part M at the second position P2 can be derived from the equations (1) to (3), and then the rotation angle of the joint of a patient can be actually obtained. For the further illustration, as shown in FIG. 5B, when a patient perform the flexion motion with the arm lifted up to a position so that the zenith angle θ′ can be derived as 45° from the measured variation of the orientation angle, the corresponding rotation angle of the shoulder joint can be found 135° by looking up FIG. 5B where the corresponding rotation angle of the joint is horizontal with the zenith angle θ′. The rest can be deduced by analogy.

Referring to FIG. 5A again, when a patient somehow can't lift the arm normally to the location represented by the solid line but to the location represented by the dashed line in FIG. 5A, the rotation angle of the shoulder joint is still 90° because the zenith angle θ′ of the moving part M lifted up represented by the dashed line and that of the moving part M normally lifted up represented by the solid line are both 90°. Besides, when the muscle of the upper arm of the patient rotates involuntarily to cause the location of the attitude sensing unit 21 to be changed, the zenith angle θ′ in such case is still the same as that in the case of normal lifting up.

FIG. 5C is another side-view of the shoulder joint measured by the joint motion measuring apparatus 2.

Different form the aspect of FIG. 5B, the vertical direction of the patient's head in FIG. 5C is defined as the negative direction of the Z axis while the downright direction of the patient's feet is defined as the positive direction of the Z axis. Accordingly, the attitude sensing unit 21 is located by the coordinates (0, 0, 1), and then the corresponding zenith angle θ′ still can be derived as 90° from the aforementioned computation, but the rotation angle thereof is equal to the zenith angle θ′. In other words, when the vertical direction of the patient's head is defined as the negative direction of the Z axis and the downright direction of the patient's feet is defined as the positive direction of the Z axis, the computed zenith angle θ′ is actually equal to the rotation angle of the joint.

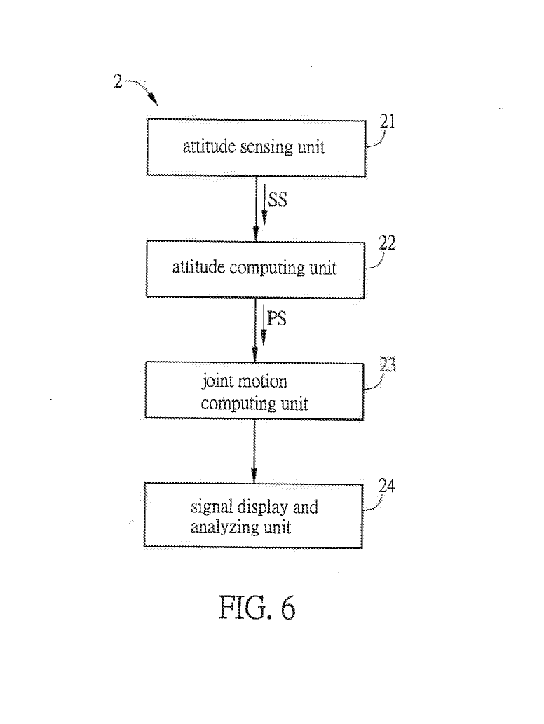

As shown in FIG. 6, the joint motion measuring apparatus 2 can further include a signal display and analyzing unit 24, which can receive the rotation angle of the joint, and display and analyze the range of motion of the joint. For example, the signal display and analyzing unit 24 can display the measurement data and provide an interactive interface to allow the mutual interaction, such as the virtual character perform the same motion as the part of the body captured by the attitude sensing unit 21. Besides, signal display and analyzing unit 24 can perform an assessment to provide the clinicians or therapists to assess the curative effect and the degree of impairment of joint movement of the patient.

Accordingly, a wearable joint motion measuring apparatus 2 is disclosed in this invention, and it can be placed on the sick limb or trunk of the patient, collecting the motion signals generated by the movement of the patient's limb or trunk. Once the patient wore the joint motion measuring apparatus 2 to perform the active joint motion, the zenith angle θ′ can be obtained through measuring the orientation angle, and then the rotation angle can be computed accordingly. To deserve to be mentioned, the wearable joint motion measuring apparatus 2 of the invention is easy to be worn, and the measurement data will not be affected even for the different wearing locations, the different measuring experiences of clinicians or therapists, and the different measuring period. Thereby, the measurement data can be more objective to provide the clinicians or therapists to effectively assess the curative effect and the degree of impairment of the patient's joint. In addition, the joint motion measuring apparatus of the invention can overcome the measurement error caused by the muscle rotation or the deviation of the movement during the measurement of the range of motion of the joint for obtaining the greater accuracy.

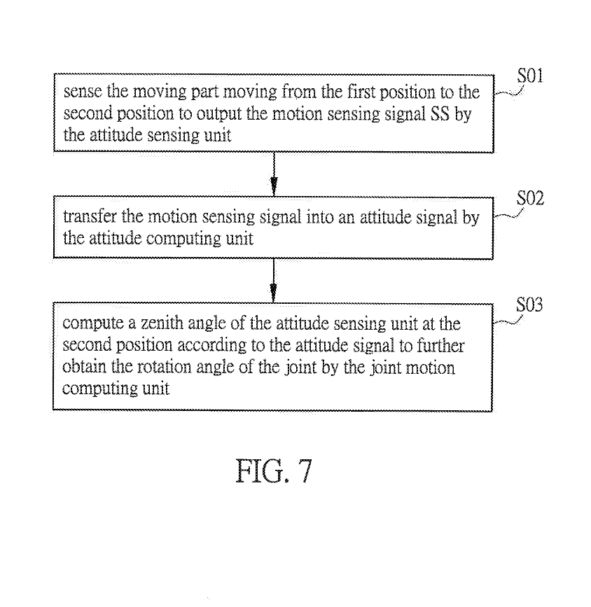

FIG. 7 is a flow chart of a joint motion measuring method according to a preferred embodiment of the invention.

Referring to FIG. 7 with FIG. 2B, the joint motion measuring method of the invention is cooperated with the joint motion measuring apparatus 2, and can measure the rotation angle of the human's joint. The human body has a moving part M connected with the joint, and the joint motion measuring apparatus 2 is wearable to be placed on the moving part M, and includes an attitude sensing unit 21, an attitude computing unit 22, and a joint motion computing unit 23.

The joint motion measuring method of the invention includes the steps S01˜S03.

The step S01 is to sense the moving part M moving from the first position P1 to the second position P2 to output the motion sensing signal SS by the attitude sensing unit 21. Herein, the motion sensing signal SS can include an angular velocity, an acceleration, a magnetic field intensity, a geomagnetic azimuth, or their any combination of the moving part M moving from the first position P1 to the second position P2.

The step S02 is to transfer the motion sensing signal SS into an attitude signal PS by the attitude computing unit 22. The attitude computing unit 22 can obtain an orientation angle according to the angular velocity, the acceleration, the magnetic field intensity, the geomagnetic azimuth, or their any combination.

The step S03 is to compute a zenith angle θ′ of the attitude sensing unit 21 at the second position P2 according to the attitude signal PS to further obtain the rotation angle of the joint by the joint motion computing unit 23. The joint motion computing unit 23 receives the orientation angle to generate a transformation matrix T. Besides, the joint motion computing unit 23 can multiply the transformation matrix T by the first position vector V1 of the attitude sensing unit 21 at the first position P1 to obtain a second position vector V2 of the attitude sensing unit 21 at the second position P2, and thus obtain the zenith angle θ′. The rotation angle is equal to the ideal angle of the range of motion of the joint minus the zenith angle θ′. Otherwise, the rotation angle is equal to the zenith angle θ′.

The other technical features of the joint motion measuring method of the invention are illustrated clearly as the above embodiments, and therefore they are not described here for concise purpose.

In summary, in the joint motion measuring apparatus and the measuring method thereof according to the invention, the attitude sensing unit senses the moving part (connected with the joint to be measured) moving from a first position to a second position to output a motion sensing signal, and the attitude computing unit transfers the motion sensing signal into an attitude signal, and then the joint motion computing unit can compute a zenith angle of the attitude sensing unit at the second position according to the attitude signal to obtain further the rotation angle of the joint. Thereby, the measurement error caused by the muscle rotation or the deviation of the movement during the measurement of the range of motion of the joint can be overcome for obtaining better accuracy.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A joint motion measuring apparatus for measuring a rotation angle of a joint of a human body having a moving part connected with the joint, comprising:
an attitude sensing unit placed on the moving part and sensing the moving part moving from a first position to a second position to output a motion sensing signal;
an attitude computing unit coupled with the attitude sensing unit and transferring the motion sensing signal into an attitude signal; and
a joint motion computing unit coupled with the attitude computing unit and computing a zenith angle of the attitude sensing unit at the second position according to the attitude signal to further obtain the rotation angle of the joint.

2. The joint motion measuring apparatus as recited in claim 1, wherein the joint motion measuring apparatus is wearable to be placed on the moving part.

3. The joint motion measuring apparatus as recited in claim 1, wherein the attitude sensing unit includes a gyroscope, an accelerometer, a magnetometer, or an electronic compass, or their any combination.

4. The joint motion measuring apparatus as recited in claim 1, wherein the motion sensing signal includes an angular velocity, an acceleration, a magnetic field intensity, a geomagnetic azimuth, or their any combination, caused by the location changes of the moving part moving from the first position to the second position.

5. The joint motion measuring apparatus as recited in claim 4, wherein the attitude computing unit integrates the angular velocities sensed by a triaxial gyroscope to obtain an orientation angle.

6. The joint motion measuring apparatus as recited in claim 4, wherein the attitude computing unit obtains an orientation angle according to the triaxial gravity components sensed by an accelerometer.

7. The joint motion measuring apparatus as recited in claim 4, wherein the attitude computing unit obtains an orientation angle according to the geomagnetic strength sensed by a triaxial magnetometer.

8. The joint motion measuring apparatus as recited in claim 4, wherein the attitude computing unit obtains an orientation angle according to a geomagnetic azimuth sensed by an electronic compass.

9. The joint motion measuring apparatus as recited in claim 4, wherein the attitude computing unit obtains an orientation angle according to the angular velocity, the acceleration, the magnetic field intensity, the geomagnetic azimuth, or their any combination.

10. The joint motion measuring apparatus as recited in claim 5, wherein the joint motion computing unit receives the orientation angle to generate a transformation matrix.

11. The joint motion measuring apparatus as recited in claim 6, wherein the joint motion computing unit receives the orientation angle to generate a transformation matrix.

12. The joint motion measuring apparatus as recited in claim 7, wherein the joint motion computing unit receives the orientation angle to generate a transformation matrix.

13. The joint motion measuring apparatus as recited in claim 8, wherein the joint motion computing unit receives the orientation angle to generate a transformation matrix.

14. The joint motion measuring apparatus as recited in claim 9, wherein the joint motion computing unit receives the orientation angle to generate a transformation matrix.

15. The joint motion measuring apparatus as recited in claim 10, wherein the joint motion computing unit multiplies the transformation matrix by a first position vector of the attitude sensing unit at the first position to obtain a second position vector of the attitude sensing unit at the second position, and thus obtain the zenith angle.

16. The joint motion measuring apparatus as recited in claim 11, wherein the joint motion computing unit multiplies the transformation matrix by a first position vector of the attitude sensing unit at the first position to obtain a second position vector of the attitude sensing unit at the second position, and thus obtain the zenith angle.

17. The joint motion measuring apparatus as recited in claim 12, wherein the joint motion computing unit multiplies the transformation matrix by a first position vector of the attitude sensing unit at the first position to obtain a second position vector of the attitude sensing unit at the second position, and thus obtain the zenith angle.

18. The joint motion measuring apparatus as recited in claim 13, wherein the joint motion computing unit multiplies the transformation matrix by a first position vector of the attitude sensing unit at the first position to obtain a second position vector of the attitude sensing unit at the second position, and thus obtain the zenith angle.

19. The joint motion measuring apparatus as recited in claim 14, wherein the joint motion computing unit multiplies the transformation matrix by a first position vector of the attitude sensing unit at the first position to obtain a second position vector of the attitude sensing unit at the second position, and thus obtain the zenith angle.

20. The joint motion measuring apparatus as recited in claim 1, wherein the rotation angle of the joint is equal to an ideal angle of the range of motion of the joint minus the zenith angle.

21. The joint motion measuring apparatus as recited in claim 1, wherein the rotation angle of the joint is equal to the zenith angle.

22. A joint motion measuring method cooperated with a joint motion measuring apparatus and applied for measuring a rotation angle of a joint of a human body having a moving part connected with the joint, wherein the joint motion measuring apparatus includes an attitude sensing unit, an attitude computing unit and a joint motion computing unit, the joint motion measuring method comprising steps of:
sensing the moving part moving from a first position to a second position to output a motion sensing signal by the attitude sensing unit;
transferring the motion sensing signal into an attitude signal by the attitude computing unit; and
computing a zenith angle of the attitude sensing unit at the second position according to the attitude signal to further obtain the rotation angle of the joint by the joint motion computing unit.

23. The joint motion measuring method as recited in claim 22, wherein the joint motion measuring apparatus is wearable to be placed on the moving part.

24. The joint motion measuring method as recited in claim 22, wherein the motion sensing signal includes an angular velocity, an acceleration, a magnetic field intensity, a geomagnetic azimuth, or their any combination, caused by the location changes of the moving part moving from the first position to the second position.

25. The joint motion measuring method as recited in claim 24, wherein the attitude computing unit obtains an orientation angle according to the angular velocity, the acceleration, the magnetic field intensity, the geomagnetic azimuth, or their any combination.

26. The joint motion measuring method as recited in claim 25, further comprising:
receiving the orientation angle to generate a transformation matrix by the joint motion computing unit.

27. The joint motion measuring method as recited in claim 26, further comprising:
multiplying the transformation matrix by a first position vector of the attitude sensing unit at the first position to obtain a second position vector of the attitude sensing unit at the second position thus to obtain the zenith angle by the joint motion computing unit.

28. The joint motion measuring method as recited in claim 22, wherein the rotation angle of the joint is equal to an ideal angle of the range of motion of the joint minus the zenith angle.

29. The joint motion measuring method as recited in claim 22, wherein the rotation angle of the joint is equal to the zenith angle.
